(19) United States
(12) Patent Application Publication
(10) Pub. No.: US 2024/0315702 A1
(43) Pub. Date: Sep. 26, 2024

(54) INTRODUCER SHEATH WITH OCCLUSION CAPABILITY

(71) Applicant: Westchester Medical Center Advanced Physician Services, P.C., Hawthorne, NY (US)

(72) Inventor: Arun Goyal, Armonk, NY (US)

(21) Appl. No.: 18/123,655

(22) Filed: Mar. 20, 2023

(51) Int. Cl. A61B 17/12; A61M 25/10

(52) U.S. Cl. CPC A61B 17/12136; A61M 25/10; A61B 2017/00557

(57) ABSTRACT

An apparatus that includes a proximal end portion, a distal end portion, and a hub disposed at the proximal end portion. The hub incudes an inflation port and a flush port. A sheath portion extends from the proximal end portion to the distal end portion and through the hub. The sheath portion includes an inner sheath that defines an inner lumen and an outer sheath that defines an outer lumen. The inner lumen is in fluid communication with the flush port and the outer lumen is in fluid communication with the inflation port. A balloon is disposed at the distal end portion and in fluid communication with the outer lumen.

### FIELD

The present disclosure is generally related medical devices, and in particular to introducer sheaths for use in medical procedures.

### BACKGROUND

A wide variety of devices have been developed for medical use. One such device is an introducer sheath that facilitates access to body lumen at an access site. Conventionally, introducer sheaths are formed of three or more components that require assembly: a sheath portion, a hub, and a hemostasis valve disposed within the hub. Introducer sheaths also commonly include flush ports to introduce saline solution, for example, into the body lumen. A suitable example of such an assembly is shown in U.S. Pat. No. 7,144,411, which depicts an introducer sheath having a construction similar to that described above, the entirety of which is hereby incorporated by reference.

In practice, introducer sheaths are often used to access a vessel or artery to allow a surgical or medical procedure to be performed. The introducer sheath is generally inserted into a patient's vasculature using the modified Seldinger technique. In the Seldinger technique, a needle is first inserted into the vessel and then followed by a guidewire through the needle. Next, the needle is removed and a sheath/dilator combination is advanced over the guidewire. The sheath/dilator expands the puncture in the vessel to a size suitable to receive the distal end of an introducer sheath. After the distal end of the sheath is disposed within the vessel, the dilator and guidewire are removed, thereby allowing access to the vessel lumen through the introducer sheath.

There are an increasing number of medical procedures that can be performed using sheaths. Medical procedures such as angioplasty, stenting, and intraaortic therapy, are examples of procedures that can include the use of introducer sheaths. In particular, the medical devices (e.g., catheters, stents, wires, balloon pumps) used in these procedures are introduced through the sheath.

Balloon occlusion catheters are also known. Such devices are used to occlude vascular lumens to prevent embolism and also used in thrombectomy procedures. An example of a balloon occlusion catheter is disclosed in U.S. Pat. No. 9,498,225, the contents of which are incorporated herein by reference in their entirety. Balloon occlusion catheters typically include a port for insertion of a balloon inflation syringe, and may also include a separate flush port.

There is a need for a vascular sheath that allows for arterial access and facilitates the insertion of wires, catheters and/or other equipment for diagnostic and vascular interventions, while also creating a vascular occlusion in case of rupture or potential rupture of the artery or other body lumen.

### SUMMARY

An objective of the present invention is to provide an apparatus that allows for insertion of equipment into body lumens of a patient, such as, for example, vascular lumens including arteries and other blood vessels, while also allowing for occlusion of and/or removal of blockage from such body lumens.

Another objective of the present invention is to provide a sheath introducer with a balloon that provides for occlusion of or removal of blockage from a body lumen through which the introducer is inserted.

An apparatus according to an exemplary embodiment of the present invention comprises: a proximal end portion; a distal end portion; a hub disposed at the proximal end portion, the hub comprising an inflation port and a flush port; a sheath portion extending from the proximal end portion to the distal end portion and through the hub, the sheath portion comprising an inner sheath that defines an inner lumen and an outer sheath that defines an outer lumen, the inner lumen being in fluid communication with the flush port and the outer lumen being in fluid communication with the inflation port; and a balloon disposed at the distal end portion and in fluid communication with the outer lumen.

In exemplary embodiments, the apparatus further comprises: a first valve; and a first line that connects the flush port to the first valve.

In exemplary embodiments, the first line comprises tubing.

In exemplary embodiments, the first valve is a stopcock valve.

In exemplary embodiments, the first valve is configured to receive liquid.

In exemplary embodiments, the apparatus further comprises: a second valve; and a second line that connects the inflation port to the second valve.

In exemplary embodiments, the second line comprises tubing.

In exemplary embodiments, the second valve is a stopcock valve.

In exemplary embodiments, the second valve is configured to receive gas injected by an inflation syringe to inflate the balloon.

In exemplary embodiments, the apparatus further comprises a hemostatic port disposed at the proximal end portion.

In exemplary embodiments, the apparatus is an introducer sheath.

### BRIEF DESCRIPTION OF THE DRAWINGS

Exemplary embodiments of the present invention will be described with references to the accompanying figures, wherein:

FIG. 1 is a perspective view of an introducer sheath according to an exemplary embodiment of the present invention;

FIG. 2 show a stopcock valve useable with the introducer sheath of FIG. 1;

FIG. 3 is a cross-sectional view of an introducer sheath according an exemplary embodiment of the present invention; and

FIGS. 4A and 4B show operation of an introducer sheath during a medical procedure in accordance with an exemplary embodiment of the present invention.

### DETAILED DESCRIPTION

The present invention is generally related to introducer sheaths, and in exemplary embodiments provides for an introducer sheath with at least two ports that allow for injection of liquids, such as medicine, contrast or other liquids or materials, while also allowing for injection of air or other gases to inflate a balloon that is part of the introducer. In exemplary embodiments, use of the apparatus during a medical procedure allows for occlusion of a body lumen (e.g., artery or other blood vessel) by inflating the balloon, administration of liquids (e.g., medicine, contrast, etc.) to the patient, and/or insertion of medical devices (e.g., guide wire, catheter, etc.) into the body lumen.

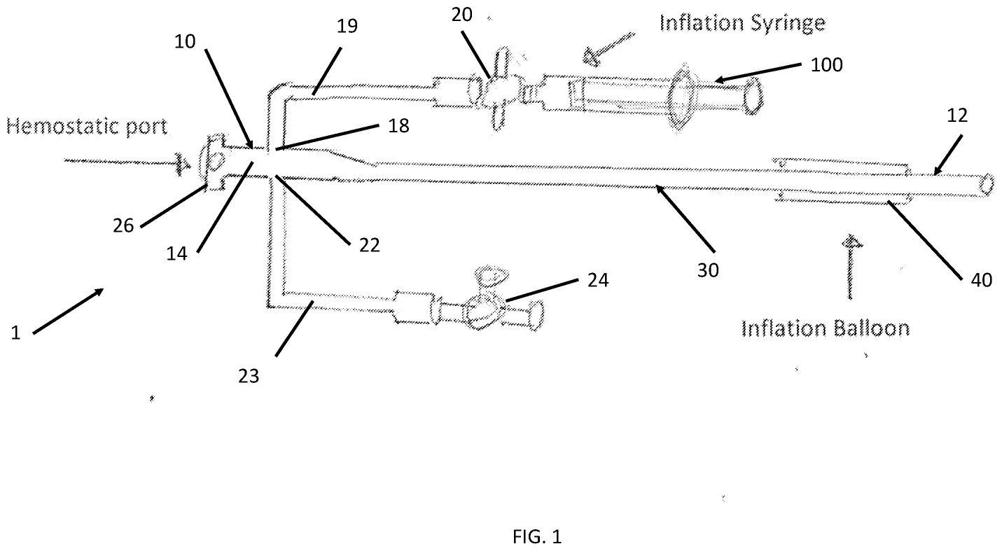

FIG. 1 shows a device, generally designated by reference number 1, according to an exemplary embodiment of the present invention. The device 1 may be considered, for example, a “introducer sheath” or “vascular device” depending on its application. The device 1 has a proximal end portion 10 and a distal end portion 12. A hub 14 is disposed at the proximal end portion 10 of the device 1. The hub includes an inflation port 18 and a flush port 22. A first line 19 connects the inflation port 18 to a first valve 20 that is configured to receive air or other gas by, for example, injection of the air or gas with an inflation syringe 100. A second line 23 connects the flush port 22 to a second valve 24 that is configured to receive fluids, such as, for example, contrast, medicine or other liquids and materials. The first and second lines 19 and 23 may be substantially transparent tubing made of polymeric material, as is known in the art. A hemostatic port 26 is disposed at the proximal end of the device 1.

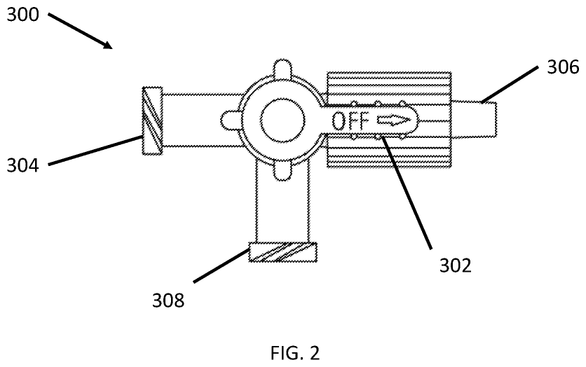

In exemplary embodiments, the first and/or second valves 20, 24 are stopcock valves. FIG. 2 shows a stopcock valve, generally designated by reference number 300, as generally known in the art and as used in various exemplary embodiments of the present invention. The stopcock valve 300 includes a rotatable knob 302, an inlet port 304, an outlet port 306, and a side port 308. Rotatable knob 302 controls the flow of fluids and materials between inlet port 304, side port 308 and an outlet port 306. The outlet port 306 is directly connected to a line extending from a corresponding port of the hub 14. As fluid flows into the inlet port 304, a user can open or close fluid flow from inlet port 304 to outlet port 306 by rotating the knob 302 in the appropriate direction, which in turn controls the fluid flow to the corresponding port of the hub 14. The user can stop the flow of fluids for a variety of reasons including preparation for removing and repositioning the introducer sheath in the vasculature of the patient, switching of infusate lines, or at the end of a procedure. When the practitioner is ready to resume the flow of fluids, the practitioner can turn rotatable knob 302 to a flow position. It should be appreciated that the first and second valves 20, 24 are not limited to stopcock valves, and other types of valves may be used in various exemplary embodiments of the present invention.

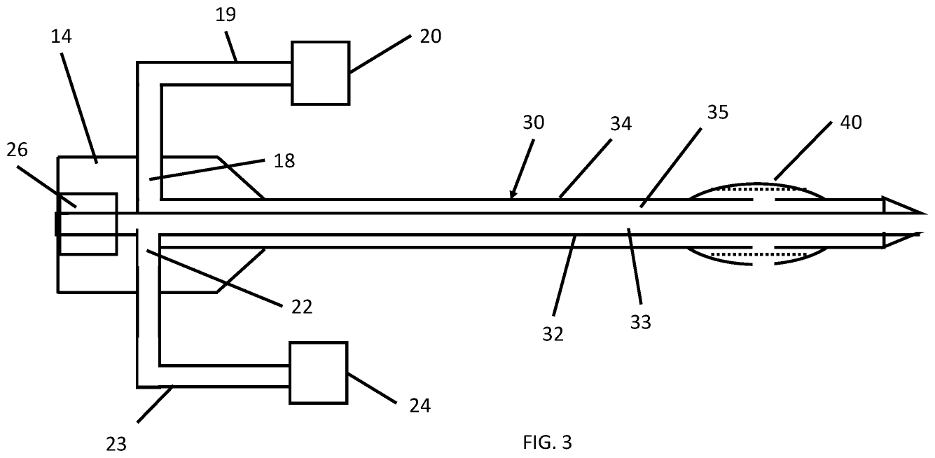

A sheath portion 30 extends through the hub 14 towards the distal end portion 12 of the device 1. As shown in FIG. 3, the sheath portion 30 includes an inner sheath 32 and an outer sheath 34. The inner sheath 32 defines an inner lumen 33 and the outer sheath 34 defines an outer lumen 35. The inner lumen 33 is in fluid communication with the second line 23, flush port 22 and the hemostatic port 26. The outer lumen 35 is in fluid communication with the first line 19 and the inflation port 18. The inner and outer sheaths 32, 34 are substantially flexible or semi-rigid tubular bodies made of, for example, polymeric material. The distal end portion of the device 12 has a size and shape to facilitate insertion into a blood vessel, and in exemplary embodiments may have a tapered tip for facilitating substantially atraumatic introduction through an incision and partial insertion into a vessel. The inner lumen 33 has a size to accommodate insertion of endoluminal devices therethrough, such as a catheter, guidewire, and the like, which may be introduced through the hemostatic port 26.

A balloon 40 is disposed around the outer sheath 34 at the distal end portion 12 of the device 1. The balloon 40 is in fluid communication with the outer lumen 35 so that the balloon may be inflated through the inflation port 18, as described in further detail below. FIG. 3 shows the balloon 40 in the inflated state, with dashed lines showing the balloon 40 in the deflated state.

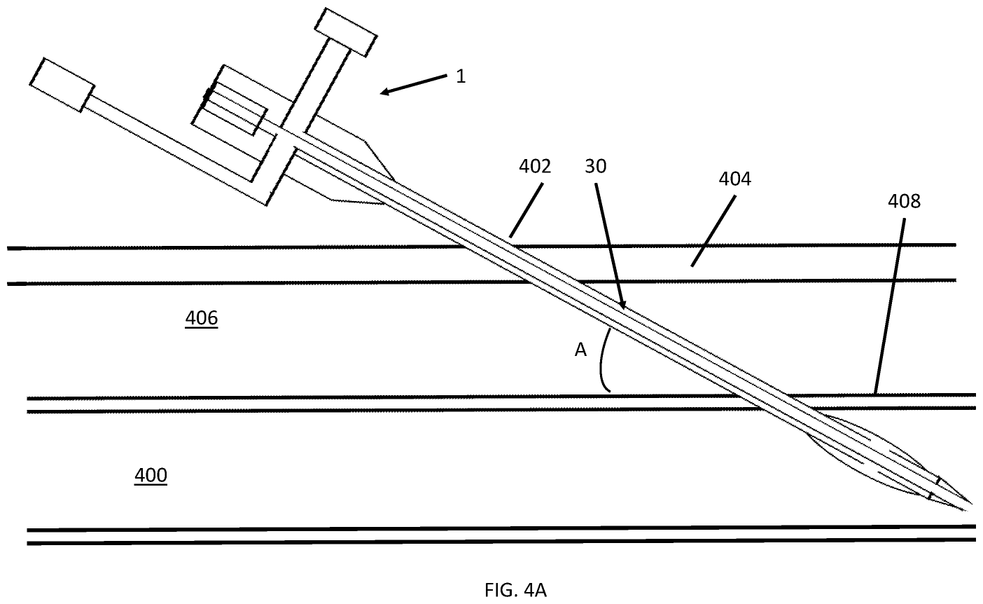

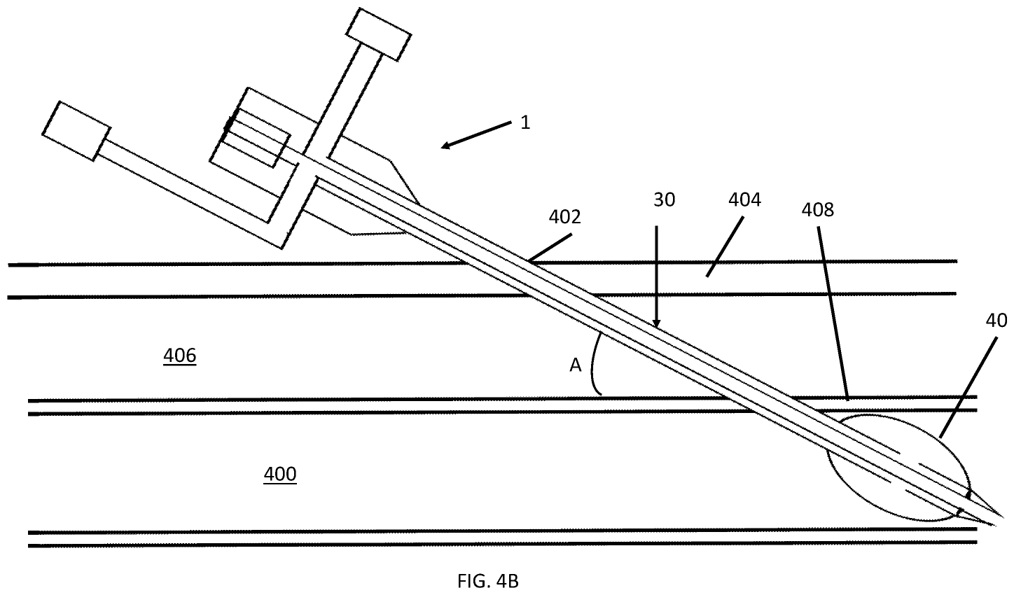

FIGS. 4A and 4B show operation of the device 1. As shown in FIG. 4A, the sheath portion 30 may be inserted or otherwise positioned within a blood vessel 400 through an incision, puncture, or other opening 402 that extends from a patient's skin 404 through any intervening tissue 406, and a wall 408 of the vessel 400. The sheath portion 30 may be advanced over a guidewire or other rail (not shown) previously positioned through the incision 402 into the blood vessel 400 using a conventional procedure. In exemplary embodiments, the blood vessel 400 is a peripheral vessel, such as a femoral or carotid artery, although other body lumens may be accessed using the sheath portion 30, as will be appreciated by those skilled in the art. The sheath portion 30 may be introduced into the vessel 400 at a variable distance based on the need of intervention.

The incision 402, and consequently the sheath portion 30, are preferably oriented at a substantially acute angle “A” with respect to the vessel 400, thereby facilitating introduction of devices through the inner lumen 33 of the inner sheath 32 into the vessel 400 with minimal risk of damage to the vessel 400. One or more devices, such as a guide wire, a catheter, and the like may be inserted into the hemostatic port 26 and through the inner sheath 32, and advanced to a desired location within the patient's body. For example, the devices may be used to perform a therapeutic or diagnostic procedure, such as angioplasty, atherectomy, stent implantation, and the like, within the patient's vasculature.

As shown in FIG. 4B, upon insertion of the sheath portion 30 into the vessel 400, or at any point during the inter-vascular procedure, the balloon 40 may be inflated. Inflation of the balloon 40 may be achieved by insertion of the inflation syringe 100 into the first valve 20 and actuation of the inflation syringe 100 to inject air through the outer lumen 35 to the balloon 40. In exemplary embodiments, the balloon 40 may be inflated to create a vascular occlusion to prevent rupture of the vessel 400 or to block a rupture that has occurred. The occluding capability of the device 1 may also be used to decrease the risk of distal embolization from vascular intervention and assist in vascular thrombectomy.

Also, at any point during the inter-vascular procedure, medicine, contrast or other liquids and materials may be introduced to the patient through the second valve 24.

While particular embodiments of the present invention have been shown and described in detail, it would be obvious to those skilled in the art that various modifications and improvements thereon may be made without departing from the spirit and scope of the invention. It is therefore intended to cover all such modifications and improvements that are within the scope of this invention.

1. An apparatus comprising:
a proximal end portion;
a distal end portion;
a hub disposed at the proximal end portion, the hub comprising an inflation port and a flush port;
a sheath portion extending from the proximal end portion to the distal end portion and through the hub, the sheath portion comprising an inner sheath that defines an inner lumen and an outer sheath that defines an outer lumen, the inner lumen being in fluid communication with the flush port and the outer lumen being in fluid communication with the inflation port; and
a balloon disposed at the distal end portion and in fluid communication with the outer lumen.

2. The apparatus of claim 1, further comprising:
a first valve; and
a first line that connects the flush port to the first valve.

3. The apparatus of claim 2, wherein the first line comprises tubing.

4. The apparatus of claim 2, wherein the first valve is a stopcock valve.

5. The apparatus of claim 2, wherein the first valve is configured to receive liquid.

6. The apparatus of claim 1, further comprising:
a second valve; and
a second line that connects the inflation port to the second valve.

7. The apparatus of claim 6, wherein the second line comprises tubing.

8. The apparatus of claim 6, wherein the second valve is a stopcock valve.

9. The apparatus of claim 6, wherein the second valve is configured to receive gas injected by an inflation syringe to inflate the balloon.

10. The apparatus of claim 1, further comprising a hemostatic port disposed at the proximal end portion.

11. The apparatus of claim 1, wherein the apparatus is an introducer sheath.
